(19) United States
(12) Patent Application Publication
(10) Pub. No.: US 2024/0225464 A1
(43) Pub. Date: Jul. 11, 2024

(54) SENSING DEVICE AND DEVICE SET

(71) Applicant: Murata Manufacturing Co., Ltd., Nagaokakyo-shi (JP)

(72) Inventors: Toru SHIMUTA, Nagaokakyo-shi (JP); Kaname HANADA, Nagaokakyo-shi (JP)

(21) Appl. No.: 18/614,898

(22) Filed: Mar. 25, 2024

Related U.S. Application Data
(63) Continuation of application No. PCT/JP22/36430, filed on Sep. 29, 2022.

(30) Foreign Application Priority Data
Oct. 5, 2021 (JP) 2021-164066

(51) Int. Cl. A61B 5/024; A61B 5/00

(52) U.S. Cl. CPC A61B 5/02433; A61B 5/6826

(57) ABSTRACT

A sensing device is provided that includes a non-flexible body that is wearable on a user's finger and a biosensor that measures biological information through the finger. The body has an inner peripheral surface facing a pad, a back, an outer side surface, and an inner side surface. The biosensor is on the inner peripheral surface to face the pad when the body is worn on the finger. In a cross-section of the inner peripheral surface, a first distance between a portion of the inner peripheral surface that faces the pad of the finger and a portion of the inner peripheral surface that faces the back of the finger is shorter than a second distance between a portion of the inner peripheral surface that faces the outer side surface of the finger and a portion of the inner peripheral surface that faces the inner side surface of the finger.

### CROSS REFERENCE TO RELATED APPLICATIONS

This application is a continuation of International Application No. PCT/JP2022/036430, filed Sep. 29, 2022, which claims priority to Japanese Patent Application No. 2021-164066, filed Oct. 5, 2021, the entire contents of each of which are hereby incorporated by reference in their entirety.

### TECHNICAL FIELD

The present invention relates to a sensing device and a device set.

### BACKGROUND

In general, a pulse wave signal can represent, as a waveform, changes in the volume of blood vessels that are caused when a heart pumps blood through the blood vessels, and a sensor that detects this volume change is referred to as a pulse wave sensor. A photoplethysmographic sensor that measures a pulse wave signal by using a photoplethysmography method has been put into practical use. The photoplethysmographic sensor includes a light-emitting element that emits light of a specific wavelength to the body surface of a user and a light-receiving element that receives light that has been reflected by or passed through the body of the user. There is a sensing device having a photoplethysmographic sensor mounted in the ring-shaped worn portion wearable on a finger of the user to measure a pulse wave signal through the finger of the user. As this type of sensing device, for example, a sensing device having a ring-shaped non-flexible worn portion with a circular cross-section or a sensing device having a ring-shaped flexible worn portion with a non-circular cross-section. International Publication No. wo 2015/068465 A1 (hereinafter “Patent Document 1”) discloses an existing sensing device having a ring-shaped flexible worn portion with a non-circular cross-section.

However, since the thickness of a human finger, which is the distance between the pad and the back of the finger, is smaller than the width of the finger, which is the distance between the outer side surface and the inner side surface of the finger, when the cross-section of the ring-shaped worn portion to be worn on the finger of the user is circular and the material of the worn portion is non-flexible, a gap is generated between the worn portion and the finger. Since the photoplethysmographic sensor does not come into close contact with the finger when such a gap is generated, the S/N ratio decreases.

On the other hand, when the material of the ring-shaped worn portion to be worn on the finger of the user is flexible, since the degree of deformation of the worn portion when worn on the finger depends on the thickness of the finger of the user, the distance between the light-emitting element and the light-receiving element of the photoplethysmographic sensor also depends on the thickness of the finger of the user. The distance between the light-emitting element and the light-receiving element of the photoplethysmographic sensor is desirably maintained at an optimal distance depending on the wavelength, regardless of the thickness of the finger of the user.

### SUMMARY OF THE INVENTION

Accordingly, the exemplary aspects of the present disclosure address the problems described above in order to improve the S/N ratio of the sensing device.

In an exemplary aspect, a sensing device is provided that includes a non-flexible body that is configured to be word on a finger of a user and has an inner peripheral surface that faces a pad, a back, an outer side surface, and an inner side surface of the finger when the body is worn on the finger; and a biosensor that is configured to measure biological information of the user through the finger. In the exemplary aspect, the biosensor is disposed on the inner peripheral surface such that the biosensor faces the pad of the finger when the body is worn on the finger, in which, in a cross-section of the inner peripheral surface, a first distance between a portion of the inner peripheral surface that faces the pad of the finger and a portion of the inner peripheral surface that faces the back of the finger is shorter than a second distance between a portion of the inner peripheral surface that faces the outer side surface of the finger and a portion of the inner peripheral surface that faces the inner side surface of the finger.

According to the configuration of the sensing device disclosed herein, close contact can be made between the pad of the finger and the biosensor in order to improve the S/N ratio of the biosensor by making the first distance shorter than the second distance.

### BRIEF DESCRIPTION OF DRAWINGS

FIG. 1 is an explanatory diagram illustrating a hardware structure of a sensing device according to an exemplary embodiment.

FIG. 2 is an explanatory diagram illustrating an external structure of a sensing device according to an exemplary embodiment.

FIG. 3 is an explanatory diagram illustrating an external structure of a sensing device according to an exemplary embodiment.

FIG. 4 is an explanatory diagram illustrating an external structure of a sensing device according to an exemplary embodiment.

FIG. 5 is an explanatory diagram illustrating a cross-sectional structure of a body of a sensing device according to an exemplary embodiment.

FIG. 6 is a partially exploded view of the sensing device according to the exemplary embodiment.

FIG. 7 is a partially enlarged view illustrating a cross-section of the body of the sensing device according to the exemplary embodiment.

FIG. 8 is a graph illustrating the measurement result of the S/N ratio versus the distance between a light-emitting element and a light-receiving element according to an exemplary embodiment.

FIG. 9 is an explanatory diagram illustrating a structure of a device set according to an exemplary embodiment.

FIG. 10 is an explanatory diagram illustrating a cross-sectional structure of the body of the sensing device according to the exemplary embodiment.

FIG. 11 is an explanatory diagram illustrating a cross-sectional structure of the body of the sensing device according to the exemplary embodiment.

FIG. 12 is an explanatory diagram illustrating a cross-sectional structure of the body of the sensing device according to the exemplary embodiment.

### DETAILED DESCRIPTION

Exemplary embodiments of the present invention will be described below with reference to the drawings. Here, the same components are denoted by the same reference numerals to emit duplicate descriptions.

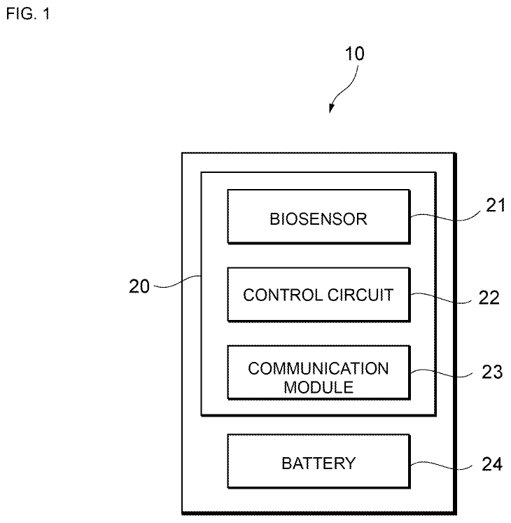

FIG. 1 is an explanatory diagram illustrating a hardware structure of a sensing device 10 according to an exemplary embodiment. The sensing device 10 includes a biosensor 21 configured to measure biological information through a finger of a user, a control circuit 22 configured to control the operation of the biosensor 21, a communication module 23 configured to transmit the measurement result of the biosensor 21 to an external computer through a wireless line or a wired circuit, and a battery 24 configured to supply electric power to the control circuit 22 and the communication module 23. The biosensor 21, the control circuit 22, and the communication module 23 are implemented in a circuit chip 20 according to an exemplary aspect.

Moreover, the biosensor 21 may include any one or more of, for example, a pulse wave sensor (photoplethysmographic sensor or piezoelectric pulse wave sensor), an oxygen saturation sensor, and a temperature sensor. For example, a reflective photoplethysmographic sensor is configured to illuminate the body surface of the user with infrared light, red light, or green light and measures the light reflected by the body surface of the user by using a photodiode or a phototransistor. Since oxygenated hemoglobin present in arterial blood absorbs incident light, a pulse wave signal can be measured by sensing, over time, the blood flow rate (e.g., change in the volume of blood vessels) that changes with the pulsation of the heart.

For example, the pulse wave feature amount can be calculated in accordance with the pulse wave signal measured by the pulse wave sensor, and the blood pressure, blood sugar level, vascular resistance, blood flow, or arteriosclerosis degree can be estimated in accordance with the pulse wave feature amount. In addition, the period of fluctuation can be obtained in accordance with the pulse wave signal, and the heart rate (e.g., pulse rate) can be estimated in accordance with the obtained period of fluctuation. In addition, the index value of the autonomic nervous function can be estimated by power spectrum analysis of frequency components of the periodic fluctuation in the heartbeat. The arterial blood oxygen saturation concentration can also be estimated by obtaining the pulsation (e.g., change amount) in accordance with the pulse wave signal measured by the oxygen saturation sensor. The body temperature of the user can be estimated in accordance with the measurement value of the temperature sensor.

According to exemplary aspects, the pulse wave sensor, the oxygen saturation sensor, and/or the temperature sensor can be used as the biosensor 21 to estimate biological information, such as the blood pressure, blood sugar level, vascular resistance, blood flow, degree of arteriosclerosis, heat rate, autonomic nervous function, arterial blood oxygen saturation concentration, or body temperature in accordance with the measurement result of the biosensor 21.

The control circuit 22 includes a processor, a memory, and an input-output interface. The control circuit 22 is configured to transmit the measurement result of the biosensor 21 to an external computer (for example, a mobile terminal, such as a multi-function mobile phone or a tablet, or a cloud server) through the communication module 23. The external computer receives the measurement result of the biosensor 21 and estimates the biological information in accordance with the received measurement result. In addition, the control circuit 22 can be configured to estimate biological information in accordance with the measurement result of the biosensor 21 without transmitting the measurement result of the biosensor 21 to the external computer.

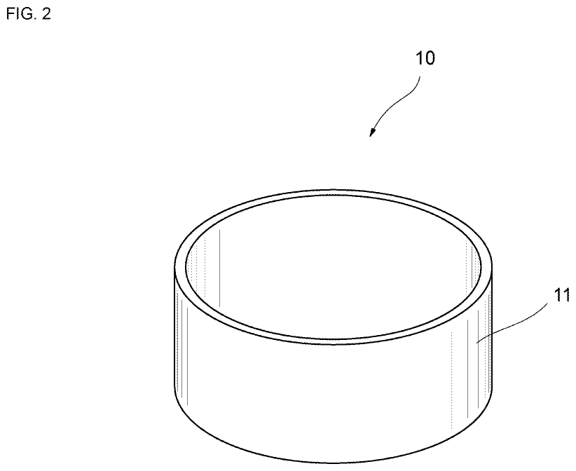

FIGS. 2 to 4 are explanatory diagrams illustrating external structures of the sensing devices 10 according to exemplary embodiments. As shown, the sensing device 10 has a ring-shaped body 11 wearable on the finger of the user.

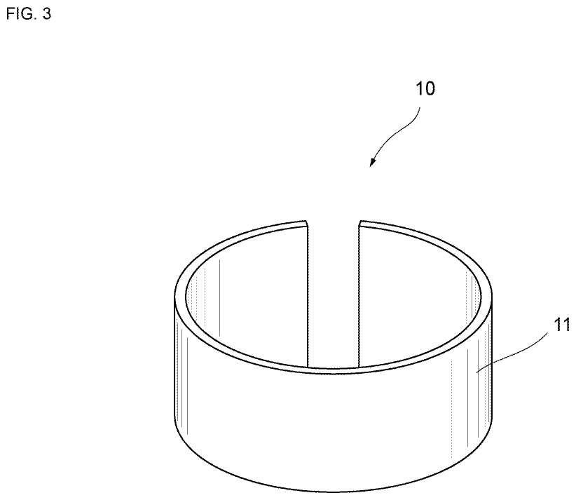

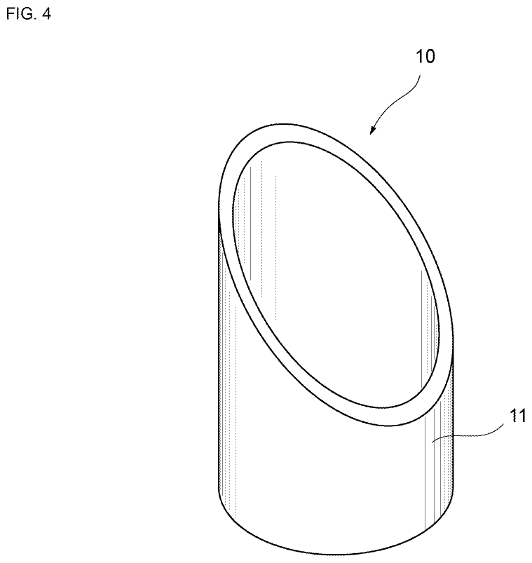

For example, in the example illustrated in FIG. 2, the body 11 has a hollow cylindrical shape. In the example illustrated in FIG. 3, a cut in an insertion-removal direction of the finger is formed in the side surface of the body 11, but the cut need not be formed. In the example illustrated in FIG. 4, the body 11 has a cylindrical shape (for example, a finger cot shape) that is configured to be fitted onto the finger of the user. The bottom (i.e., portion with which the fingertip comes into contact) of the cylinder may be present or absent in various exemplary aspects.

According to the exemplary aspect, the body 11 can be formed of a non-flexible material (for example, metal, ceramic, glass, hard resin). It is noted that the property of non-flexibility is an anti-bending property against an external force that maintains the original shape. Specifically, for example, with the body worn on the finger of the user, the body does not deform due to an external force applied when the finger is bent. In addition, when the body 11 is made of a non-flexible material, the design and durability is improved as compared with a flexible material, such as rubber or sponge. As a result, the user can easily wear the sensing device 10 at all times in daily life.

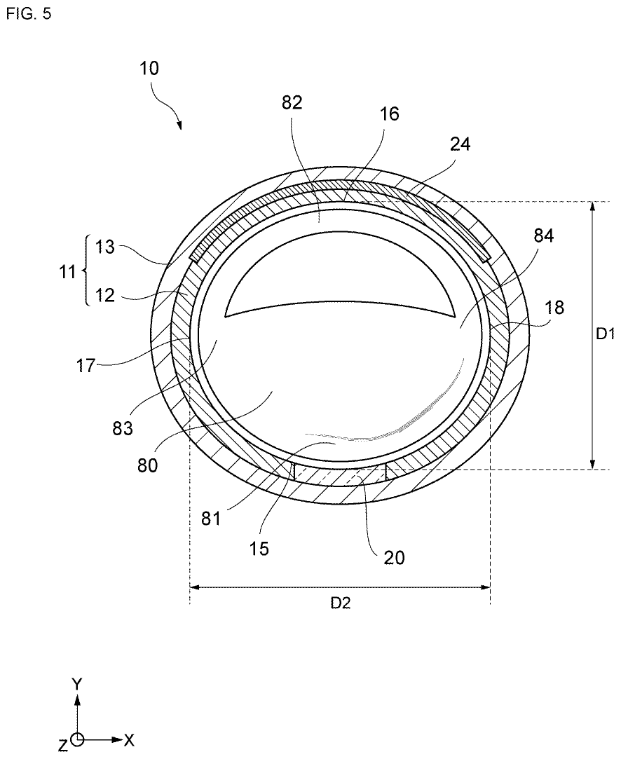

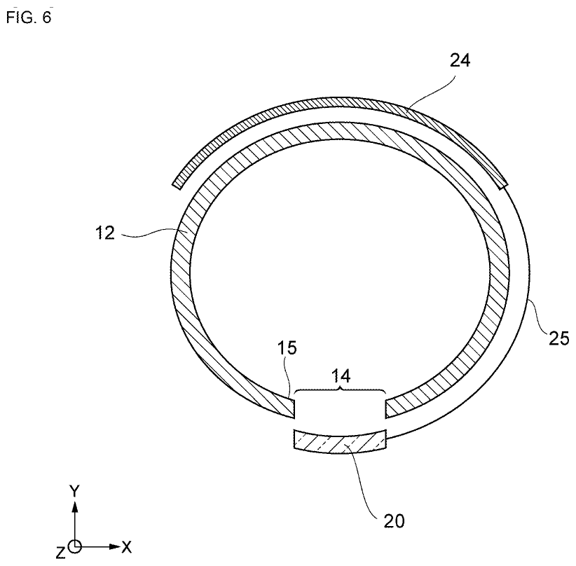

FIG. 5 is an explanatory diagram illustrating a cross-sectional structure of the body 11 of the sensing device 10 according to an exemplary embodiment. For purposes of this disclosure, an X-direction and a Y-direction in the drawing are orthogonal to the insertion-removal direction of a finger 80 of the user, and the Z-direction is parallel to the insertion-removal direction of the finger 80 of the user. Moreover, the cross-section of the body 11 in the drawing is parallel to the X-direction and the Y-direction and orthogonal to the Z-direction, which is noted to be similar in FIGS. 6, 7, 9, and 10. FIG. 6 is a partially exploded view of the body 11 without the finger disposed therein.

As illustrated in FIG. 5, the body 11 includes an inner peripheral surface 12 and an outer peripheral surface 13 that have a hollow annular shape. Of two surfaces forming the inner peripheral surface 12, the surface facing the finger 80 is referred to as a front surface, and a surface facing away from the front surface is referred to as a back surface according to the exemplary aspect. The front surface of the inner peripheral surface 12 includes portions 15, 16, 17, and 18 that face the pad 81, the back 82, the outer side surface 83, and the inner side surface 84, respectively, of the finger 80 when the body 11 is worn on the finger 80. As illustrated in FIG. 6, a cavity (e.g., a hole) 14 that passes through the front and the back of the inner peripheral surface 12 is formed in the portion 15 facing the pad 81 of the finger 80, and the circuit chip 20 is fitted into the cavity 14 from the back surface of the inner peripheral surface 12. After being fitted into the cavity 14, the circuit chip 20 is fixed to the inner peripheral surface 12 with an adhesive or an adhesive tape, for example. By the circuit chip 20 being fitted into and fixed in the cavity 14, the position of the circuit chip 20 can be secured and thus prevented from deviating when an external force is applied to the circuit chip 20. Moreover, the battery 24 is mounted on the back surface of the inner peripheral surface 12. Reference numeral 25 denotes a wiring cable that connects the battery 24 and the circuit chip 20 to each other. The wiring cable 25 is also mounted on the back surface of the inner peripheral surface 12 (the wiring cable 25 is not illustrated in FIG. 5). The biosensor 21 mounted in the circuit chip 20 is disposed on the inner peripheral surface 12 to face the pad 81 of the finger 80.

It general, it should be understood that the pad 81 of the finger 80 is relatively softer than the back 82 and has more blood vessels. Since the biological information described above is measured in accordance with blood or blood vessels, the S/N ratio of the biosensor 21 can be increased by the biosensor 21 being disposed on the inner peripheral surface 12 to face (e.g., it comes into close contact) the pad 81 of the finger 80. In particular, when the pulse wave feature amount is calculated in accordance with the pulse wave signal and the blood pressure, blood sugar level, vascular resistance, blood flow, or arteriosclerosis is estimated in accordance with the pulse wave feature amount, the biosensor 21 needs to have a high S/N ratio, and accordingly, the biosensor 21 should be disposed to face (e.g., come into close contact with) the pad 81 of the finger 80.

In a cross-section of the inner peripheral surface 12, a first distance D1 between a portion 15 of the inner peripheral surface 12 that faces the pad 81 of the finger 80 and a portion 16 of the inner peripheral surface 12 that faces the back 82 of the finger 80 is shorter than a second distance D2 between a portion 17 of the inner peripheral surface 12 that faces the outer side surface 83 of the finger 80 and a portion 18 of the inner peripheral surface 12 that faces the inner side surface 84 of the finger 80. The cross-sectional shape of the inner peripheral surface 12 is, for example, substantially elliptical in the exemplary aspect.

When the distance between the pad 81 and the back 82 of the finger 80 is defined as the thickness of the finger 80, and the distance between the outer side surface 83 and the inner side surface 84 of the finger 80 is defined as the width of the finger 80, the statistical average value of the ratio of the thickness to the width of the finger 80 is approximately 0.93 for both men and women. It is noted that the term “approximately” as used herein is provided to take into account minor manufacturing variances, for example.

Moreover, it is noted that if the first distance D1 and the second distance D2 are set to a single value (i.e., the same value), a gap is generated between the pad 81 of the finger 80 and the biosensor 21, which leads to a decrease of the S/N ratio of the biosensor 21. Accordingly, when the first distance D1 is shorter than the second distance D2 (for example, the ratio of the first distance D1 to the second distance D2 is set to equal to the statistical average of the ratio of the thickness to the width of the finger 80), close contact can be ensured between the pad 81 of the finger 80 and the biosensor 21. This configuration improves the S/N ratio of the biosensor 21. Preferably, the ratio of the first distance D1 to the second distance D2 is, for example, 0.85 to 0.95 in an exemplary aspect.

It is noted that, since the Y-direction is parallel to the thickness direction of the inner diameter of the inner peripheral surface 12, the first distance D1 is the maximum value of the inner diameter of the inner peripheral surface 12 in the thickness direction. Since the X-direction is parallel to the width direction of the inner diameter of the inner peripheral surface 12, the second distance D2 is the maximum value of the inner diameter of the inner peripheral surface 12 in the width direction.

According to an exemplary aspect, a projection in contact with the finger 80 can be formed in any one of the portions 15, 16, 17, and 18 of the inner peripheral surface 12 that face the pad 81, the back 82, the outer side surface 83, and the inner side surface 84, respectively, of the finger 80 or the vicinity thereof. In this case, the first distance D1 denotes the distance between the portion 15 of the inner peripheral surface 12 that faces the pad 81 of the finger 80 and the portion 16 of the inner peripheral surface 12 that faces the back 82 of the finger 80. In addition, the second distance D2 denotes the distance between the portion 17 of the inner peripheral surface 12 that faces the outer side surface 83 of the finger 80 and the portion 18 of the inner peripheral surface 12 that faces the inner side surface 84 of the finger 80 with the projection removed.

When the first distance D1 is shorter than the second distance D2, a slight gap is generated between the outer side surface 83 of the finger 80 and the portion 17 of the inner peripheral surface 12 that faces the outer side surface 83 and between the inner side surface 84 of the finger 80 and the portion 18 of the inner peripheral surface 12 that faces the inner side surface 84, but the pressure on the finger 80 can be relaxed because the flesh of the finger 80 that is pushed out when the finger 80 is bent escapes to the gap on the side.

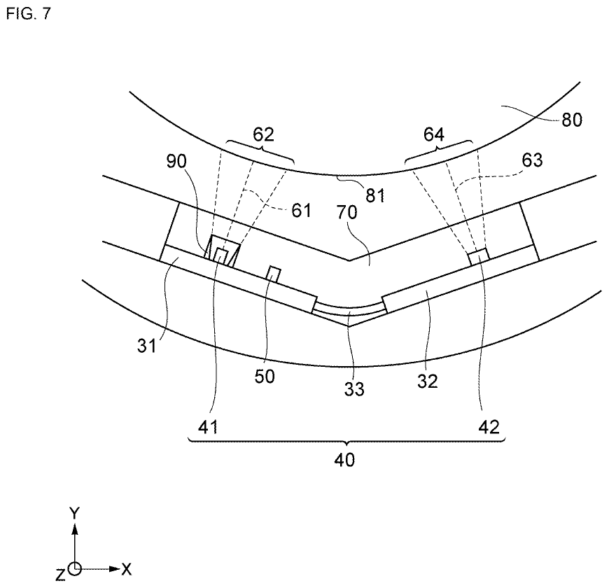

FIG. 7 is a partially enlarged view illustrating a cross-section of the body 11 of the sensing device 10 according to the exemplary embodiment. According to an exemplary aspect, an example in which a reflective photoplethysmographic sensor 40 and a temperature sensor 50 are used as the biosensor 21 will be described.

In particular, the photoplethysmographic sensor 40 includes a light-emitting element 41 and a light-receiving element 42. For example, a semiconductor laser, such as a vertical cavity surface emitting laser, a light emitting diode, or the like can be used as the light-emitting element 41. For example, a photodiode, a phototransistor, or the like can be used as the light-receiving element 42. When the cross-sectional shape of the inner peripheral surface 12 is, for example, substantial elliptical, the short axis of the ellipse is a line segment that connects the portion 15 of the inner peripheral surface 12 that faces the pad 81 of the finger 80 and the portion 16 of the inner peripheral surface 12 that faces the back 82 of the finger 80. The light-emitting element 41 and the light-receiving element 42 may be disposed symmetrically with respect to, for example, the short axis of the substantial ellipse. For example, when the wavelength of light from the light-emitting element 41 is, for example, in a wavelength range around near-infrared light, the distance between the light-emitting element 41 and the light-receiving element 42 is preferably, for example, 10 mm, which may be approximately the same as the width of the finger. By disposing the light-emitting element 41 and the light-receiving element 42 symmetrically with respect to, for example, the short axis of the substantial ellipse, a pulse wave can be measured on the pad of the finger even when the subject has a short finger.

In addition, the circuit chip 20 includes two rigid substrates 31 and 32, a flexible substrate 33 that connects these two rigid substrates 31 and 32 to each other, and a resin layer 70 in which the rigid substrates 31 and 32 and the flexible substrate 33 are encapsulated. The light-emitting element 41 and the temperature sensor 50 are mounted on the rigid substrate 31 in this exemplary aspect. Moreover, the light-receiving element 42 is mounted on the rigid substrate 32. It is noted that the control circuit 22 and the communication module 23 may be mounted on the rigid substrates 31 and 32 or on other substrates.

The photoplethysmographic sensor 40 is desirably brought into close contact with the finger 80 during measurement of a pulse wave signal to improve the S/N ratio of the photoplethysmographic sensor 40. The reason why the S/N ratio of the photoplethysmographic sensor 40 decreases when a gap is generated between the photoplethysmographic sensor 40 and the finger 80 will be described below.

According to an exemplary aspect, the material of the resin layer 70 can be, for example, an epoxy resin, a silicone resin, an acrylic resin, a polycarbonate resin, or a polyethylene terephthalate resin, and the refractive index thereof is approximately 1.4 to 1.6. For convenience of description, the refractive index of the resin layer 70 is assumed to be 1.5, the refractive index of the skin of the finger 80 is assumed to be 1.3, and the refractive index of air is assumed to be 1. Here, when the refractive index of the media on the incident side is assumed to be n1 and the refractive index of the media on the transmission side is assumed to be n2, the reflective index R of light incident orthogonally on the interface between these two media is R=(n2−n1)²/(n2+n1)².

When there is no gap between the photoplethysmographic sensor 40 and the finger 80, the reflective index at the interface between the resin layer 70 and the finger 80 is 0.005 (transmittance is 0.995). When there is a gap between the photoplethysmographic sensor 40 and the finger 80, the reflective index at the interface between the resin layer 70 and air is 0.040 (transmittance is 0.960), and the reflective index at the interface between air and the finger 80 is 0.017 (transmittance is 0.983). As a result, when there is a gap between the photoplethysmographic sensor 40 and the finger 80, the transmittance of the two interfaces described above is 0.960×0.983≈0.944, which is 94.9% of the transmittance when there is no gap between the photoplethysmographic sensor 40 and the finger 80.

Since the transmittance of light emitted from the light-emitting element 41 and the transmittance of light received by the light-receiving element 42 when there is a gap between the photoplethysmographic sensor 40 and the finger 80 are 94.9% of the transmittance of light emitted from the light-emitting element 41 and the transmittance of light received by the light-receiving element 42 when there is no gap between the photoplethysmographic sensor 40 and the finger 80, the amount of light received by the photoplethysmographic sensor 40 when there is a gap between the photoplethysmographic sensor 40 and the finger 80 decreases to approximately 90% of the amount of light received by the photoplethysmographic sensor 40 when there is no gap between the photoplethysmographic sensor 40 and the finger 80.

It is further noted that, since the reflective index and the transmittance of light also depend on the angle of incidence of light on the interface and the thickness of a gap, when a gap is generated between the photoplethysmographic sensor 40 and the finger 80, the amount of light received by the photoplethysmographic sensor 40 greatly changes and the S/N ratio decreases.

According to the exemplary embodiment, when the first distance D1 is shorter than the second distance D2, close contact can be made between the pad 81 of the finger 80 and the biosensor 21 even when the body 11 is formed of a non-flexible material and to improve the S/N ratio of the biosensor 21.

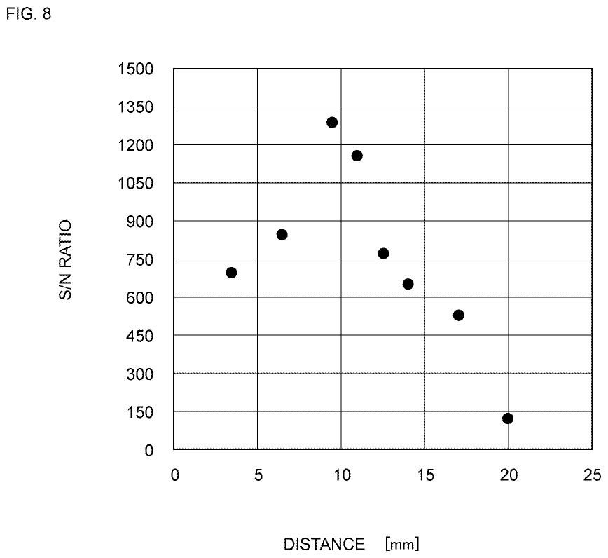

The distance between the light-emitting element 41 and the light-receiving element 42 is desirably set within an optimal range depending on the wavelength of light emitted from the light-emitting element 41. For example, when biological information of a deep region of the skin of the finger 80 need be measured, a wavelength (for example, 940 nm) around near-infrared light is suitable. FIG. 8 is a graph illustrating the measurement result of the S/N ratio versus the distance between the light-emitting element that emits near-infrared light and the light-receiving element that receives near-infrared light. Since the signal intensity of a photoelectric pulse wave signal is pulse wave amplitude and changes depending on respiration and environmental temperature even for a single subject, there may be slight variations in measurement, but the S/N ratio is maximized when the distance between the light-emitting element and the light-receiving element is approximately 10 mm. In a wavelength region around near-infrared light, the optimal distance between the light-emitting element and the light-receiving element is considered to be approximately 10 mm. When the distance between the light-emitting element and the light-receiving element deviates from the optimal distance, the S/N ratio decreases.

On the other hand, for a wavelength range of green light, which has high bioabsorbability than near-infrared light and red light, the optimal distance between the light-emitting element and the light-receiving element is considered to be approximately 2 to 3 mm.

It is also noted that an oxygen saturation sensor includes a light-emitting element that emits near-infrared light, a light-emitting element that emits red light, and a light-receiving element that receives near-infrared light and red light. When the ratio of the alternating current component to the direct current component of a photoelectric pulse wave signal of red light is (AC/DC)_{Red}, and the ratio of the alternating current component to the direct current component of a photoelectric pulse wave signal of near-infrared light is (AC/DC)_{IR}, the oxygen saturation is calculated by [(AC/DC)_{Red}]/[(AC/DC)_{IR}]. Since the ratio of the alternating current component of the photovoltaic pulse wave signal to the direct current component changes when the distance between the light-emitting element and the light-receiving element changes, the distance between the light-emitting element that emits near-infrared light and the light-receiving element is desirably set to the same value as the distance between the light-emitting element that emits red light and the light-receiving element.

According to an exemplary aspect, the distance between the light-emitting element 41 and the light-receiving element 42 is shorter than the distance (that is, the width of the finger 80) between the outer side surface 83 and the inner side surface 84 of the finger 80. When the distance between the light-emitting element 41 and the light-receiving element 42 is longer than the width of the finger 80, the length of the optical path of light passing through the inside of the finger 80 changes depending on the thickness of the finger 80, performance, such as the S/N ratio, may vary. When the distance between the light-emitting element 41 and the light-receiving element 42 is shorter than the width of the finger 80, the length of the optical path of light passing through the inside of the finger 80 becomes constant, and performance, such as the S/N ratio, is stabilized.

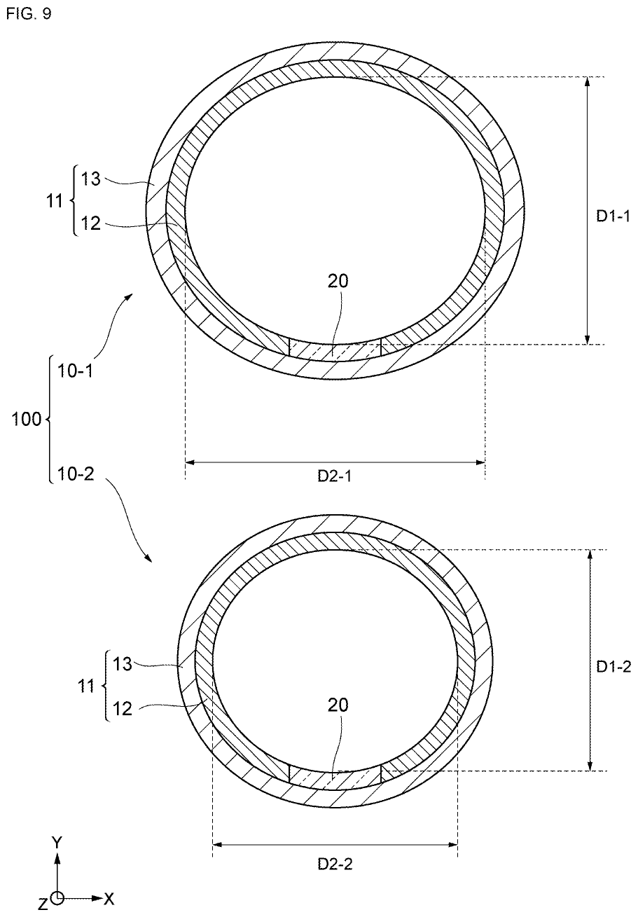

FIG. 9 is an explanatory diagram illustrating a structure of a device set 100 according to an exemplary embodiment. The device set 100 includes a plurality of sensing devices 10-1 and 10-2. The sensing devices 10-1 and 10-2 have different reference numerals to distinguish between them, but the basic structures of the sensing devices 10-1 and 10-2 are similar to the structure of the sensing device 10.

The sensing device 10-1 is designed for users (for example, men) with a thick finger 80, and a first distance D1-1 and a second distance D2-1 of the inner peripheral surface 12 are set to be longer. On the other hand, the sensing device 10-2 is designed for users (for example, women) with a thin finger 80, and a first distance D1-2 and a second distance D2-2 of the inner peripheral surface 12 are set to be shorter. Since the first distance D1-1 and the second distance D2-1 of the inner peripheral surface 12 of the sensing device 10-1 are designed differently from the first distance D1-2 and the second distance D2-2 of the inner peripheral surface 12 of the sensing device 10-2 in accordance with the thickness of the finger of the user who uses the sensing device 10-1, close contact can be made between the finger 80 and the biosensor 21.

However, the distance between the light-emitting element 41 and the light-receiving element 42 that form the biosensor 21 of the sensing device 10-1 is desirably set to be identical to the distance between the light-emitting element 41 and the light-receiving element 42 that form the biosensor 21 of the sensing device 10-2. The performance of the biosensor 21 can be identical between the plurality of sensing devices 10-1 and 10-2 by setting the distance between the light-emitting element 41 and the light-receiving element 42 constant (that is, setting the distance within an optimal distance range in accordance with the wavelength), regardless of the thickness of the finger of the user who uses the sensing device, as described above.

It is also noted that, for convenience of description, FIG. 9 illustrates an example in which the number of sensing devices forming the device set 100 is two, but the number of sensing devices forming the device set 100 may be three or more in alternative aspects. In this case, the first distance and the second distance of the inner peripheral surface of one sensing device of the plurality of sensing devices may differ from the first distance and the second distance of the inner peripheral surface of another sensing device of the plurality of sensing devices. However, the distance between the light-emitting element 41 and the light-receiving element 42 that form the biosensor 21 of each of the sensing devices is identical.

Referring back to FIG. 7, in a wavelength region around near-infrared light, the optimal distance between the light-emitting element 41 and the light-receiving element 42 is considered to be approximately 10 mm. On the other hand, the minimum width of the finger 80 for Japanese women is approximately 14.1 mm. Taking into consideration that the skin surface of the finger 80 is curved, when the light-emitting element 41 is disposed on a plane at a distance of approximately 10 mm from the light-receiving element 42, contact between the pulse wave sensor 40 and the finger 80 decreases. In view of these circumstances, the light-emitting element 41 and the light-receiving element 42 are bent and arranged on the inner peripheral surface 12 to face the pad 81 of the finger 80. For example, since the flexible substrate 33 that connects the rigid substrates 31 and 32 to each other is bent (e.g., into a valley) in the example illustrated in FIG. 7, the light-emitting elements 41 and the light-receiving element 42 can be disposed on the inner peripheral surface 12 to face the bent portion of the pad 81 of the finger 80. The shape of the bent arrangement may be, for example, a V-shape or a U-shape according to exemplary aspects. When the distance between the light-emitting element 41 and the light-receiving element 42 is set to be approximately 10 mm, the depth of the V-shaped valley is desirably approximately 1 to 2.5 mm.

With the configuration that the light-emitting element 41 and the light-receiving element 42 are disposed on the inner peripheral surface 12 to face the bent portion of the pad 81 of the finger 80, a maximum emission direction 61 of the light-emitting element 41 and a maximum reception direction 63 of the light-receiving element 42 can be aligned with directions orthogonal to the surface of the pad 81 of the finger 80. By the light-emitting element 41 being disposed such that the maximum emission direction 61 of the light-emitting element 41 is orthogonal to the surface of the pad 81 of the finger 80, the ratio of light that reaches the blood vessels in the dermis of the finger 80 can be increased. Similarly, by the light-receiving element 42 being disposed such that the maximum reception direction 63 of the light-receiving element 42 is orthogonal to the surface of the pad 81 of the finger 80, the ratio of light that is reflected by blood vessels in the dermis of the finger 80 and reaches the light-receiving element 42 can be increased. It is noted that, depending on the thickness of the finger 80, the maximum emission direction 61 of the light-emitting element 41 may deviate from a direction orthogonal to the surface of the pad 81 of the finger 80, or the maximum reception direction 63 of the light-receiving element 42 may deviate from the direction orthogonal to the surface of the pad 81 of the finger 80. However, the deviation can be decreased by the light-emitting element 41 and the light-receiving element 42 being bent and arranged as described above.

Since the light-emitting element 41 and the light-receiving element 42 are bent and arranged on the inner peripheral surface 12 to face the bent portion of the pad 81 of the finger 80, the maximum emission direction 61 of the light-emitting element 41 and the maximum reception direction 63 of the light-receiving element 42 are not parallel to each other.

Yet further, the resin layer 70 can be bent to fit the curved shape of the flexible substrate 33 such that the surface of the resin layer 70 is orthogonal to the maximum emission direction 61 of the light-emitting element 41 and the maximum reception direction 63 of the light-receiving element 42. By the resin layer 70 being bent such that the surface of the resin layer 70 is orthogonal to the maximum emission direction 61 of the light-emitting element 41, light emitted from the light-emitting element 41 can be suppressed from being refracted on the surface of the resin layer 70, and the transmittance of the emitted light can be suppressed from being decreased. Similarly, by the resin layer 70 being bent such that the surface of the resin layer 70 is orthogonal to the maximum reception direction 63 of the light-receiving element 42, light received by the light-receiving element 42 can be suppressed from being refracted on the surface of the resin layer 70, and the transmittance of the received light can be suppressed from being decreased.

It is also noted that instead of the mounting method illustrated in FIG. 7, the light-emitting element 41 and the light-receiving element 42 may be mounted on the flexible substrate, and the flexible substrate may be bent at a position between the light-emitting element 41 and the light-receiving element 42. In addition, a flexible cable can be used instead of the flexible substrate 33 in an alternative aspect.

The resin layer 70 is desirably formed integrally by using a metal mold to integrally encapsulate the rigid substrates 31 and 32 and the flexible substrate 33. Since a deviation in the relative positional relationship (distance, angle, and the like between both elements) of the light-emitting element 41 and the light-receiving element 42 can be suppressed by the resin layer 70 being formed integrally to integrally encapsulate the rigid substrates 31 and 32 and the flexible substrate 33, a state in which the S/N ratio of the photoplethysmographic sensor 40 is optimal can be maintained.

According to an exemplary aspect, the electrode terminals of the light-emitting element 41, the light-receiving element 42, and other circuit elements are preferably not exposed through the resin layer 70. As a result, the daily life waterproof of the sensing device 10 is achieved.

The light-emitting element 41 is surrounded by a reflector 90 in an exemplary aspect. When the light-emitting element 41 is surrounded by the reflector 90, light emitted from the light-emitting element 41 can be suppressed from being directly incident on the light-receiving element 42 without being incident on the finger 80, thereby improving the S/N ratio of the photoplethysmographic sensor 40. The light-receiving element 42 may be surrounded by reflector 90 while the light-emitting element 41 is surrounded by the reflector.

The resin layer 70 is desirably formed such that the end portions and the corner portions of the resin layer 70 are located outside the range of the directional angle 62 of the light-emitting element 41, and the end portions and the corner portions of the resin layer 70 are located outside the range of the directional angle 64 of the light-receiving element 42. This configuration prevents light emitted from the light-emitting element 41 being directly incident on the light-receiving element 42 after being dispersed or reflected by the end portions and the corner portions of the resin layer 70 without being incident on the finger 80.

In addition, the light-emitting element 41 is desirably disposed on the inner peripheral surface 12 such that, when the body 11 is worn on the finger 80, the pad 81 of the finger 80 is located within the range of the directional angle 62 of the light-emitting element 41. This configuration suppresses light emitted from the light-emitting element 41 from being directly incident on the light-receiving element 42 after being reflected by the body 11 without being incident on the finger 80.

In addition, the light-receiving element 42 is desirably disposed on the inner peripheral surface 12 such that the pad 81 of the finger 80 is located within the range of the directional angle 64 of the light-receiving element 42 when the body 11 is worn on the finger 80. This configuration suppresses the light reflected by the body 11 without being incident on the finger 80 from being directly incident on the light-receiving element 42.

It is noted that, in addition to the structure described above, as a structure for suppressing stray light, a structure can be provided that includes, for example, a resin layer in which the light-emitting element 41 is encapsulated separately from a resin layer in which the light-receiving element 42 is encapsulated and includes a black resin that divides the two resin layers.

Moreover, a thermistor can be used as the temperature sensor 50 in an exemplary aspect. Since air has a low thermal conductivity, no gap is desirably present between the finger 80 and the temperature sensor 50 to accurately measure the temperature (e.g., an obliteration temperature) of the finger 80. In addition, the thermal resistance between the temperature sensor 50 and the finger 80 is desirably low, and the thermal resistance between the temperature sensor 50 and the outside air is desirably high. In view of these circumstances, the temperature sensor 50 is desirably encapsulated in the resin layer 70. The thermal conductivity of the resin layer 70 is three orders lower than that of metal and one order higher than that of air. The thermal resistance between the temperature sensor 50 and the finger 80 can be decreased by adjusting the thickness of the resin layer 70 between the temperature sensor 50 and the finger 80 to a value smaller than, for example, 1 mm. In addition, the thermal resistance between the temperature sensor 50 and the outside air can be increased by appropriately adjusting the shape and the thickness of the body 11.

The photoplethysmographic sensor 40 including the light-emitting element 41 and the light-receiving element 42 has been exemplified in the above description, but the photoplethysmographic sensor 40 may include a plurality of light-emitting elements that emit light of different wavelengths and a light-receiving element in an alternative exemplary aspect. In particular, the plurality of light-emitting elements can include, for example, a light-emitting element that is configured to emit light of red to near-infrared wavelengths and a light-emitting element that is configured to emit light of blue to yellow green wavelengths. Red to near-infrared wavelengths are suitable to measure biological information from a deep region of the skin of the finger 80. On the other hand, blue to yellow green wavelengths are suitable to measure biological information from a shallow region of the skin of the finger 80. Thus, in this configuration, biological information can be measured from a deep region of the skin of the finger 80 and a shallow region of the skin by using both the light-emitting element that emits light of red to near-infrared wavelengths and the light-emitting element that emits light of blue to yellow green wavelengths.

Furthermore, the plurality of light-emitting elements can be disposed at positions that differ in distance from the light-receiving element depending on the wavelengths. For example, a light-emitting element configured to emit light of red to near-infrared wavelengths can be disposed at a distance of approximately 10 mm from the light-receiving element, and a light-emitting element configured to emit light of blue to yellow green wavelengths can be disposed at a distance of approximately 2 to 3 mm from the light-receiving element. In this configuration, biological information can be measured at a stable S/N ratio by the plurality of light-emitting elements being disposed at positions that differ in distance from the light-receiving element depending on the wavelengths to measure.

It is also noted that the photoplethysmographic sensor 40 may include a plurality of light-emitting elements that emit light of different wavelengths and a plurality of light-receiving elements that receive light of different wavelengths. The photoplethysmographic sensor 40 may include, for example, a first pair having a first light-emitting element configured to emit light of red to near-infrared wavelengths and a first light-receiving element configured to receive light from the first light-emitting element and a second pair having a second light-emitting element configured to emit light of blue to yellow green wavelengths and a second light-receiving element configured to receive light from the second light-emitting element. In this case, the first light-emitting element can be disposed at a distance of approximately 10 mm from the first light-receiving element, and the second light-emitting element can be disposed at a distance of approximately 2 to 3 mm from the second light-receiving element.

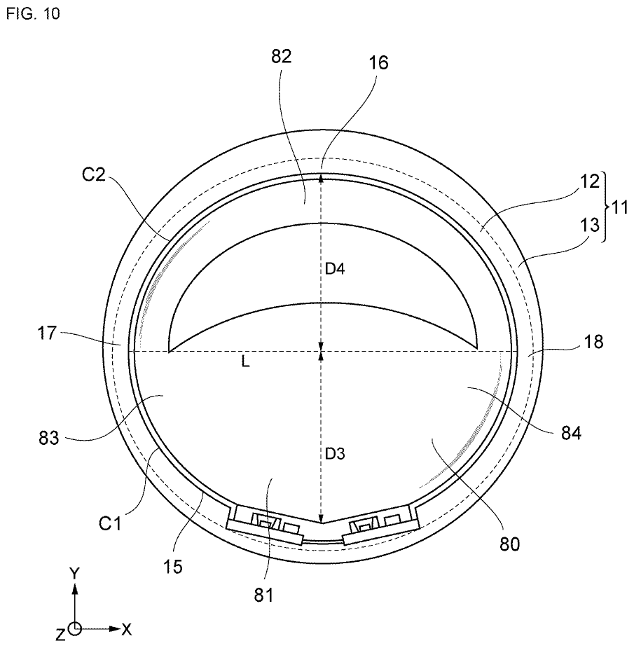

FIG. 10 is an explanatory diagram illustrating an example of a cross-sectional structure of the body 11 of the sensing device 10 according to the exemplary embodiment. In the example in this drawing, a first curvature radius is defined by the curvature radius of a first curve C1 that passes through the portion 17 of the inner peripheral surface 12 that faces the outer side surface 83 of the finger 80, the portion 15 of the inner peripheral surface 12 that faces the pad 81 of the finger 80, and the portion 18 of the inner peripheral surface 12 that faces the inner side surface 84 of the finger 80. In addition, a second curvature radius is defined by the curvature radius of a second curve C2 that passes through the portion 17 of the inner peripheral surface 12 that faces the outer side surface 83 of the finger 80, the portion 16 of the inner peripheral surface 12 that faces the back 82 of the finger 80, and the portion 18 of the inner peripheral surface 12 that faces the inner side surface 84 of the finger 80. Moreover, the first curve C1 and the second curve C2 define the cross-sectional shape of the inner peripheral surface 12. In this configuration, the first curvature radius is greater than the second curvature radius.

In the structure described above, since an appropriate gap is generated between the first curve C1 of the inner peripheral surface 12 and the finger 80, when the finger 80 is bent with the finger inserted into the hollow portion of the inner peripheral surface 12, the flesh of the finger 80 easily escapes into this gap, and the finger 80 can be easily bend. If the second curvature radius is greater than the first curvature radius, the upper portion of the second joint of the finger 80 gets caught on the inner peripheral surface 12 and the finger 80 is less likely to be removed from the inner peripheral surface 12. In addition, since there is almost no gap between the portion of the first curve C1 of the inner peripheral surface 12 and the finger 80, when the finger 80 is bent with the finger 80 inserted into the hollow portion of the inner peripheral surface 12, the flesh of the finger 80 does not sufficiently escape into this gap and the finger 80 cannot be bent.

It is also noted that the first curve C1 may be a portion of a circle having a first curvature, and the second curve C2 may be a portion of a circle having a second curvature. In this aspect, the first curvature is smaller than the second curvature. That is, the cross-sectional shape of the inner peripheral surface 12 may be a combination of a plurality of portions of circles having different curvatures. It is also noted that the cross-sectional shape of the inner peripheral surface 12 is not limited to a circle, an ellipse, or a combination thereof, and the curvature radius of the curve defining the cross-sectional shape of the inner peripheral surface 12 can change continuously.

It is further noted that in the example illustrated in FIG. 10, when a straight line that defines the maximum width of the inner peripheral surface 12 is assumed to be L, a distance D3 between the straight line L and the portion 15 of the inner peripheral surface 12 is shorter than a distance D4 between the straight line L and the portion 16 of the inner peripheral surface 12.

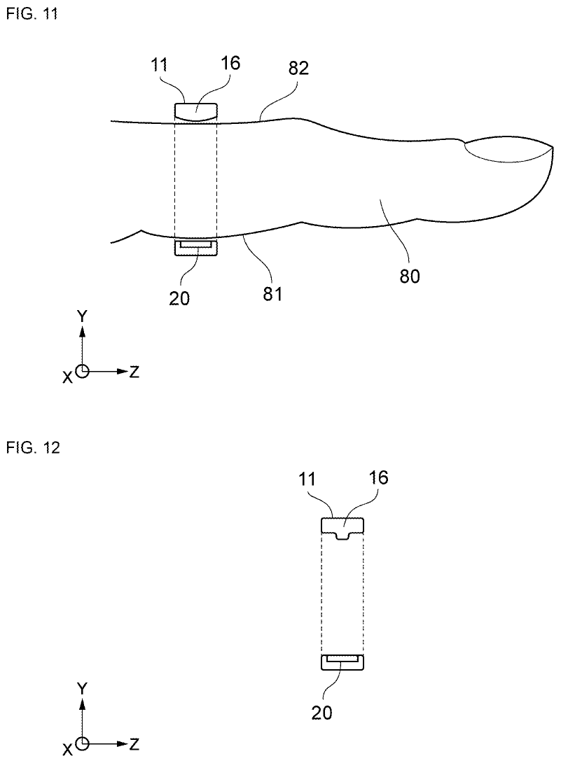

FIG. 11 is an explanatory diagram illustrating an example of a cross-sectional structure of the body 11 of the sensing device 10 according to the exemplary embodiment. In the drawing, the direction of a line segment connecting the portion 17 of the inner peripheral surface 12 that faces the outer side surface 83 of the finger 80 to the portion 18 of the inner peripheral surface 12 that faces the inner side surface 84 of the finger 80 is assumed to be the X-direction, the direction of a line segment connecting the portion 15 of the inner peripheral surface 12 that faces the pad 81 of the finger 80 to the portion 16 of the inner peripheral surface 12 that faces the back 82 of the finger 80 is assumed to be the Y-direction, and the insertion-removal direction of the finger 80 (i.e., the longitudinal direction of the finger 80) of the user is assumed to be the Z-direction. At this time, the cross-sectional shape of the portion 16 of the inner peripheral surface 12 that faces the back 82 of the finger 80 taken along a plane parallel to the YZ plane and orthogonal to the X-direction projects toward the hollow portion. In the example illustrated in the drawing, the cross-sectional shape of the portion 16 of the inner peripheral surface 12 taken along a plane parallel to the YZ plane and orthogonal to the X-direction is gently curved convexly toward the hollow portion. In the structure described above, the contact area between the back 82 of the finger 80 and the portion 16 of the inner peripheral surface 12 can be decreased, and sliding between the back 82 of the finger 80 and the portion 16 of the inner peripheral surface 12 becomes smooth when the sensing device 10 is inserted or removed, and accordingly, the sensing device 10 can be easily inserted or removed.

It is further be noted that in the example illustrated in FIG. 11, the cross-sectional shape of the portion 16 of the inner peripheral surface 12 taken along a plane parallel to the YZ plane and orthogonal to the X-direction is curved convexly toward the hollow portion with rounded corners, but, as illustrated in FIG. 12, the cross-sectional shape of the portion 16 of the inner peripheral surface 12 taken along a plane parallel to the YZ plane and orthogonal to the X-direction may project like a rectangle toward the hollow portion with sharp corners in an alternative aspect.

Finally, it is noted that the exemplary embodiments described above are intended to facilitate understanding of the present disclosure and are not intended to limit the present invention. The present invention may be modified or improved without departing from the spirit thereof, and the present invention also includes equivalents thereof. That is, appropriate design changes made to the embodiment by those skilled in the art are also included within the scope of the present invention as long as the changed embodiment has the characteristics of the present invention. In addition, it should be appreciated that elements according to the exemplary embodiment can be combined with each other within a technically possible range, and combinations of these elements are also included within the scope of the present invention as long as they include the characteristics of the exemplary aspects of the present disclosure.

### REFERENCE SIGNS LIST

10 sensing device
11 body
12 inner peripheral surface
13 outer peripheral surface
14 cavity
20 circuit chip
21 biosensor
22 control circuit
23 communication module
50 temperature sensor
80 finger
81 pad
82 back
83 outer side surface
84 inner side surface

1. A sensing device comprising:
a non-flexible body configured to be worn on a finger of a user and having an inner peripheral surface that faces a pad, a back, an outer side surface, and an inner side surface of the finger when the body is worn on the finger; and
a biosensor configured to measure biological information of the user through the finger, the biosensor being disposed on the inner peripheral surface in a position to face the pad of the finger when the body on the finger,
wherein, in a cross-section of the inner peripheral surface, a first distance between a portion of the inner peripheral surface that faces the pad of the finger and a portion of the inner peripheral surface that faces the back of the finger is shorter than a second distance between a portion of the inner peripheral surface that faces the outer side surface of the finger and a portion of the inner peripheral surface that faces the inner side surface of the finger.

2. The sensing device according to claim 1, wherein a cross-sectional shape of the inner peripheral surface is a substantial ellipse.

3. A device set comprising:
a plurality of sensing devices that each include the sensing device according to claim 1,
wherein the first distance and the second distance of the inner peripheral surface of a first sensing device of the plurality of sensing devices differ from the first distance and the second distance of the inner peripheral surface of a second sensing device of the plurality of sensing devices,
wherein the biosensor includes a pulse wave sensor including a light-emitting element and a light-receiving element, and
wherein a distance between the light-emitting element and the light-receiving element is identical for each of the first and second sensing devices.

4. The sensing device according to claim 1, wherein:
the biosensor includes a pulse wave sensor including a light-emitting element and a light-receiving element, and
a distance between the light-emitting element and the light-receiving element is shorter than a distance between the outer side surface of the finger and the inner side surface of the finger.

5. The sensing device according to claim 1, wherein:
the biosensor includes a pulse wave sensor including a light-emitting element and a light-receiving element, and
the light-emitting element and the light-receiving element are bent relative to each other and configured on the inner peripheral surface to face the pad of the finger when the body on the finger.

6. The sensing device according to claim 5, wherein:
a cross-sectional shape of the inner peripheral surface is a substantial ellipse, and
the light-emitting element and the light-receiving element are disposed symmetrically with respect to a short axis of the substantial ellipse.

7. The sensing device according to claim 5, wherein a distance between the light-emitting element and the light-receiving element is approximately 10 mm.

8. The sensing device according to claim 7, wherein the light-emitting element is configured to emit light of red to near-infrared wavelengths.

9. The sensing device according to claim 5, wherein a maximum emission direction of the light-emitting element and a maximum reception direction of the light-receiving element are not parallel to each other.

10. The sensing device according to claim 1, wherein the biosensor includes a pulse wave sensor that includes including a light-emitting element, a light-receiving element, and a resin layer that encapsulates the light-emitting element and the light-receiving element.

11. The sensing device according to claim 10, wherein the resin layer is configured such that an end portion and a corner portion of the resin layer are located outside a range of a directional angle of the light-emitting element, or the end portion and the corner portion of the resin layer are located outside a range of a directional angle of the light-receiving element.

12. The sensing device according to claim 11, wherein:
the light-emitting element is disposed on the inner peripheral surface such that the pad of the finger is located within the range of the directional angle of the light-emitting element when the body is worn on the finger, and
the light-receiving element is disposed on the inner peripheral surface such that the pad of the finger is located within the range of the directional angle of the light-receiving element when the body is worn on the finger.

13. The sensing device according to claim 12, wherein the light-emitting element and the light-receiving element are disposed in a cavity of the inner peripheral surface.

14. The sensing device according to claim 1, wherein the biosensor includes a temperature sensor.

15. The sensing device according to claim 1, wherein the biosensor includes a pulse wave sensor including a plurality of light-emitting elements that are configured to emit light of different wavelengths, respectively, and a light-receiving element, and the plurality of light-emitting elements are disposed at positions that differ in distance from the light-receiving element depending on the wavelengths, respectively.

16. The sensing device according to claim 15, wherein the plurality of light-emitting elements includes a second light-emitting element that is configured to emit light of blue to yellow green wavelengths.

17. The sensing device according to claim 16, wherein a distance between the second light-emitting element and the light-receiving element is 2 to 3 mm.

18. The sensing device according to claim 1,
wherein a first curvature radius is defined by a curvature radius of a curve that passes through a portion of the inner peripheral surface that faces the outer side surface of the finger, a portion of the inner peripheral surface that faces the pad of the finger, and a portion of the inner peripheral surface that faces the inner side surface of the finger,
wherein a second curvature radius is defined by a curvature radius of a curve that passes through a portion of the inner peripheral surface that faces the outer side surface of the finger, a portion of the inner peripheral surface that faces the back of the finger, and a portion of the inner peripheral surface that faces the inner side surface of the finger, and
the first curvature radius is greater than the second curvature radius.

19. The sensing device according to claim 1, wherein the inner peripheral surface has a hollow shape.

20. The sensing device according to claim 19, wherein, when a direction of a line segment that connects the portion of the inner peripheral surface that faces the outer side surface of the finger to the portion of the inner peripheral surface that faces the inner side surface of the finger is an X-direction, a direction of a line segment that connects the portion of the inner peripheral surface that faces the pad of the finger to the portion of the inner peripheral surface that faces the back of the finger is an Y-direction, and a direction in which the finger is inserted is a Z-direction, a cross-sectional shape of the portion of the inner peripheral surface that faces the back of the finger taken along a plane that is parallel to an YZ plane and orthogonal to the X-direction projects to the hollow portion.
